(19) United States
(12) Patent Application Publication
(10) Pub. No.: US 2010/0286043 A1
(43) Pub. Date: Nov. 11, 2010

(54) USE OF NEUREGULIN-1 IN REDUCING BRAIN DAMAGE

(75) Inventors: Christiane E.L. Dammann, Newton, MA (US); Olaf Dammann, Newton, MA (US)

Correspondence Address: NUTTER MCCLENNEN & FISH LLP, SEAPORT WEST, 155 SEAPORT BOULEVARD, BOSTON, MA, 02210-2604, US

(73) Assignee: Tufts Medical Center, Inc., Boston, MA (US)

(21) Appl. No.: 12/681,707

(22) PCT Filed: Oct. 6, 2008
(86) PCT No.: PCT/US2008/078995

§ 371 Date: Jul. 9, 2010

Related U.S. Application Data
(60) Provisional application No. 60/977,750, filed on Oct. 5, 2007.

(51) Int. Cl. A61K 38/18; G01N 33/68; C12Q 1/68; A61P 25/00

(52) U.S. Cl. 514/9.6; 436/86; 435/6

(57) ABSTRACT

Methods of reducing and/or protecting against disorders in perinatal subjects are disclosed. Such methods can be used for disorders associated with neuronal cell damage. In certain aspects, the method comprises administering a therapeutically effective amount of neuregulin or a biologically active analog with a pharmaceutical carrier to a perinatal subject. In addition, the perinatal subject can be a fetus where the neuregulin is administered to the pregnant mother. Methods for assessing whether a perinatal subject is at risk for developing a neurological disorder are also disclosed. For example, expression levels of neuregulin can be used as an indication the perinatal subject is at risk for developing a disorder associated with neuronal cell damage. Evaluating the genotype of the NRG locus in a perinatal subject can also be used as an indicator of the risk of developing neurological disorders.

### CROSS REFERENCE TO RELATED APPLICATIONS

The present application claims the benefit of U.S. Provisional Application No. 60/977,750 filed on Oct. 5, 2007, entitled “Use of Neuregulin-1 in Reducing Brain Damage.” The entire contents of the provisional application are hereby incorporated herein by reference.

### GOVERNMENT SUPPORT

The invention was supported by National Institutes of Health Grant No.: 5K08HL004436-05. The government has certain rights in the invention.

### FIELD OF THE INVENTION

The present invention concerns therapeutic agents for treatment of neurological conditions and, in particular, therapeutic agents for reducing the risk of brain damage in perinatal subjects.

### BACKGROUND OF THE INVENTION

Nearly a half of a million infants are born prematurely (less than 37 weeks gestation) each year, according to the report released by the National Center for Health Statistics (NCHS). The prematurity rate has increased nearly every year since 1981. These pre-term infants often present neurodevelopmental deficits which comprise global cognitive delay, cerebral palsy, blindness, and deafness.

While the cause of most cases of white matter damage in newborns is unknown, infants born at or before 28 weeks gestation appear to be at highest risk of brain white matter damage (WMD, 18-36%) which in turn is associated with an elevated risk of later neurologic and cognitive limitations. A common example of white matter injury observed in infants as a complication of premature birth is referred to as periventricular leukomalacia (PVL). PVL is the principal neuropathological correlate of cerebral palsy. PVL is characterized by focal necrosis of the deep periventricular white matter involving all cellular components, combined with a more diffuse white matter injury that appears selective for developing oligodendrocytes (OLs). Reduced cerebral myelin is the most prominent subsequent cerebral abnormality observed in premature infants with evidence of PVL in the neonatal period.

Deficits such as cognitive delay and cerebral palsy may be attributed, at least in part, to both endogenous and exogenous components. Exogenous components could be infection/inflammation, hypoxia-ischemia, and excitotoxicity in white and/or gray matter of the brain. One of the endogenous components is the paucity of developmentally regulated protectors, such as neuregulin.

Neuregulins (NRGs) play important roles during fetal brain, heart and lung development, and are involved in inflammatory damage processes. There are several NRG isoforms, all produced by alternative splicing of the same region on human chromosome 8p22 and all containing an EGF-like domain. One of these isoforms, NRG-1β, appears to help signal the onset of surfactant synthesis in the fetal lung. There is also increasing evidence for a protective function in experimental brain ischemia and in the etiology of several adult neurologic diseases. A number of recent reports have shown that administration of NRG-1 reduces delayed ischemic cortical damage following transient middle cerebral artery occlusion when administered before the onset of ischemia in adult rats.

However these studies fail to address the potentially protective factor of neuregulin in the neonatal brain.

### SUMMARY OF THE INVENTION

In one aspect, the invention discloses a method of reducing and/or protecting against disorders associated with neuronal cell or oligodendrocyte (OL) damage in a perinatal subject in need thereof, comprising administering a therapeutically effective amount of neuregulin (NRG) and a pharmaceutically acceptable carrier to the perinatal subject. The invention can also comprise administering neuregulin-1 (NRG-1), one of the proteins within the neuregulin family. The perinatal subject of the invention can be a neonate and/or a fetus. In one embodiment, the perinatal subject can be a neonate born prior to 32 weeks of gestation. In some instances where the subject is a fetus, neuregulin can be delivered to a pregnant mother such that therapeutically effective amounts of neuregulin can be administered to the fetus. Alternatively, therapeutically effective amounts of neuregulin can be delivered directly to the fetus. In another embodiment, the method further comprises increasing expression of ErbB-receptors as a method to increase activation of specific signaling pathways. NRG and its ErbB receptors can influence the growth and maturation of immature oligodendrocytes, and maturation can be disordered when a full complement of NRG protein and receptor are not available. Thus NRG induced activation and orchestration of ErbB signaling pathways can be accomplished through increasing ErbB-receptor expression and activation of distinct ErbB signaling pathways for potential interaction with the administered neuregulin.

In one embodiment, neuregulin can be administered in conjunction with an endogenous and/or exogenous protector. In some embodiments, the co-administration of neuregulin with at least one endogenous and/or exogenous protector can produce synergistic effects. Non-limiting examples of exogenous protectors that can be used in conjunction with the present invention comprise caffeine and/or indomethacine. Endogenous protectors can be, for example, one or more glucocorticoids and thyroid hormones. Non-limiting examples of glucocorticoids that can be used in this invention comprise dexamethasone, hydrocortisone, alclometasone, amcinonide, beclometasone, betamethasone, budesonide, ciclesonide, clobetasol, clobetasone, clocortolone, cloprednol, cortivazol, deflazacort, deoxycorticosterone, desonide, desoximetasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, rimexolone, tixocortol, triamcinolone, ulobetasol.

The administration of the therapeutically effective amount of NRG can be at least one of an oral administration, a parenteral administration, an intravenous administration, an intramuscular administration, an intraamniotic administration, a sub-cutaneous administration, a transdermal administration, an intratechal administration, a rectal administration, intravaginal administration, intra peritoneal or amniotic administration, and an intranasal administration.

In another aspect, the invention can provide a method of assessing whether a perinatal subject is at risk for developing a neurological disorder associated with neuronal cell damage that comprises evaluating levels of neuregulin. In the instances where neuregulin levels are reduced level or lacking, the perinatal subject can be at risk for developing the disorder. The perinatal subject of the invention can be a neonate and/or a fetus. In one embodiment, the perinatal subject can be a neonate born prior to 32 weeks of gestation.

In one embodiment the invention can comprise measuring expression levels of neuregulin and can further comprise measuring expression levels of ErbB receptors when evaluating the risk of developing a neurological disorder. In another embodiment, measuring expression levels of neuregulin can comprise measuring levels of at least one of a ribonucleic acid, a deoxyribonucleic acid and a protein. In addition, the invention can also comprise analyzing the neuregulin gene for one or more single nucleotide polymorphism where one or more polymorphism can be SNP8NRG221533, a polymorphism in the NRG gene that can lead to a low production of NRG protein.

In another embodiment, the invention can comprise evaluating activation levels of neuregulin signaling pathways. Since neuregulin can stimulate the tyrosine phosphorylation of ErbB receptors and activate downstream signal transduction pathways, activation levels can be influenced by genetic modifications that exist in ErbB receptor genetic loci. In one embodiment, the step of evaluating levels further comprises analyzing at least one ErbB receptor gene for one or more single nucleotide polymorphism. In some embodiments the neurological disorders can be at least one of cerebral palsy, mental retardation and leaning disabilities.

In another aspect, the invention provides a method of evaluating a risk of a NRG-1 deficiency in a perinatal subject that comprises assaying a sample from the perinatal subject for a polymorphism associated with decreased expression of NRG-1. In some embodiments, the perinatal subject can be a neonate and/or a fetus. In yet another embodiment, the sample to be assayed can be obtained from a fetus in a pregnant subject, or the pregnant subject itself.

In another aspect, the invention provides a method of evaluating risk of a neuregulin deficiency in a perinatal subject that comprises providing a nucleic acid sample from the perinatal subject, determining a single nucleotide polymorphism (SNP) genotype of neuregulin and comparing the SNP genotype with a predetermined SNP genotype whereby the perinatal subject is predicted to be at risk of a neuregulin deficiency if the SNP genotype comprises at least one of a SNP8NRG221132, a SNP8NRG221533, a SNP8NRG241930, a SNP8NRG243177 and a SNP8NRG433E1006.

In another aspect, the invention provides a method of diagnosing or predicting risk of a neuregulin deficiency in a perinatal subject comprising determining a presence or absence of a neuregulin polymorphism where the polymorphism is at least one of a SNP8NRG221132, a SNP8NRG221533, a SNP8NRG241930, a SNP8NRG243177 and a SNP8NRG433E1006. In one embodiment, determining the presence or absence of a polymorphism comprises enzymatic amplification of nucleic acid from the perinatal subject. Furthermore, the enzymatic amplification can be a step of a polymerase chain reaction protocol. Another embodiment can utilize restriction fragment length polymorphism analysis to determine the polymorphism. In some aspects of the invention, sequence analysis can be used to determine the presence or absence of the polymorphism. In yet one more embodiment, the method comprises determining the presence of a SNP8NRG221533 polymorphism.

### DETAILED DESCRIPTION OF THE INVENTION

Given the lack of methods for treating pre-term infants with neurodevelopmental deficits where neuregulin's effect on the developing brain is missing or deficient, a need exists in the art for new methods of reducing and/or preventing brain damage in neonates. In particular, new methods capable of reducing white brain matter damage would satisfy a long-felt therapeutic need.

The terms used in this invention are, in general, expected to adhere to standard definitions generally accepted by those having ordinary skill in the art of neurobiology. A few exceptions, as listed below, have been further defined within the scope of the present invention. Other terms are defined explicitly elsewhere in the present application, implicitly through the context of their usage in the present application, and/or having their ordinary meaning as understood by those skilled in the art.

The terms “neuronal” and “neural” are used interchangeably and pertain to the cells of the brain, spinal cord and peripheral nerve cord (nervous system). Examples of cells of the nervous system comprise but are not limited to: neurons, oligodendrocytes, astrocytes, ependymal cells, microglia, satellite cells and Schwann cells.

The terms “disorders associated with neuronal cell damage,” “disorders associated with neuronal cell death,” and “neurological disorder” refer to an impairment or absence of a normal neurological function or presence of an abnormal neurological function in a subject. Such disorders comprise disorders characterized by injury and/or death to brain cells, including white matter cells and/or gray matter cells. For example, neurological disorders can be the result of disease, endogenous, or exogenous injury. As used herein, neurological disorder also comprises neurodegeneration and chronic inflammation which causes morphological and/or functional abnormality of a neural cell or a population of neural cells.

Non-limiting examples of morphological and functional abnormalities comprise physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Such disorders comprise, but are not limited to, periventricular leukomalacia (PVL), cerebral palsy, mental retardation, stroke, epilepsy, seizures, dementia, memory loss, and attention deficit disorder (ADD).

Neurological disorders also comprise neurodegenerative diseases. Neurodegeneration can occur in any area of the brain of a subject and can be seen with many disorders comprising, but not limited to, Amyotrophic Lateral Sclerosis (ALS), multiple sclerosis, Huntington's disease, Parkinson's disease and Alzheimer's disease. Further neurological disorders comprise CNS (central nervous system) damage resulting from infectious diseases such as viral encephalitis, bacterial or viral meningitis and CNS damage from tumors. The neuroprotective and/or neural regenerative strategy of the present invention can also be used to improve the cell-based replacement therapies used to treat or prevent various demyelinating and dysmyelinating disorders, such as Pelizaeus-Merzbacher disease, multiple sclerosis, various leukodystrophies, post-traumatic demyelination, and cerebrovasuclar accidents. Disorders of the central nervous system further comprise mental disorders such as mood disorders (i.e., depression, bipolar disorder), anxiety disorders, autism spectrum disorders, memory disorders and schizophrenic disorders. In addition, the present invention can also find use in enhancing the cell-based therapies used to repair damaged spinal cord tissue following a spinal cord injury.

The term “mental retardation” is intended to refer to sub-average intellectual ability that is equivalent to or less than an IQ of 70, can be present from birth or infancy, and can be manifested by abnormal development, by learning difficulties, or by problems in social adjustment. Mental retardation can often be observed in preterm infants, particularly those of low birth weight and gestational age.

The term “neonatal stroke” refers to sudden diminution or loss of consciousness, sensation, and voluntary motion caused by rupture or obstruction (as by a clot) of an artery of the brain which occurs in a baby up to one year old.

The term “cerebral palsy” is intended to refer to a disability resulting from damage to the brain before, during or shortly after birth and outwardly manifested by muscular coordination and speech disturbances. Cerebral palsy can be caused by damage to one or more specific areas of the brain during development. The causes of cerebral palsy are diverse and comprise genetic, metabolic, infectious, traumatic, endocrine, and hypoxic/ischemic disorders. As used herein, the terms “hypoxic” and “hypoxia” refer to a deficiency of oxygen reaching the tissues of the body. The terms “ischemic” and “ischemia”, as used herein, refer to localized tissue anemia due to obstruction of the inflow of arterial blood.

The term “excitotoxicity” is art recognized and is intended to refer to increased levels of excitatory amino acids which are toxic to neurons. This may occur when protective mechanisms are inhibited by ischemia or inflammation.

The terms “gray matter injury” and “white matter injury” are intended to refer to injury to the gray or white matter of the brain. Such injury comprises injury to brain cells (e.g., white matter injury) and brain cell death (e.g., white matter cell death and myelin basic protein loss). As used herein, “gray matter” refers to neural tissue of the brain that contains cell bodies as well as nerve fibers, has a brownish gray color, and can form most of the cortex and nuclei of the brain. As used herein, “white matter” refers to neural tissue that consists largely of myelinated nerve fibers, has a whitish color, and underlies the gray matter of the brain.

The term “PVL” is intended to refer to certain type of damage and softening of the white matter, the inner portion of the brain that transmits information between the nerve cells and the spinal cord, as well as from one part of the brain to another. PVL is characterized by focal necrosis with a loss of all cellular elements deep in the periventricular white matter, and diffuse white matter involvement characterized by injury to glial cells which are thought to be oligodendrocyte precursors.

The terms “neonate” and “neonatal” are intended to refer to infants up to one year old. In the context of the present invention, the neonates are typically preterm infants. As used herein, the term “preterm” is intended to refer to a baby born at a gestational age of less than 266 days or 38 weeks.

The terms “fetus” and “fetal” are intended to refer to a developing human from approximately three months after conception to birth.

The term “perinatal” is intended to refer to the period shortly before and after birth, art defined as beginning with completion of 20-28 weeks of gestation and ending 7 to 28 days after birth.

The terms “neuregulin,” “NRG,” “neuregulin-1” and “NRG-1” are used interchangeably to refer to proteins or peptides that can bind and activate ErbB3 or ErbB4 protein kinases, such as all neuregulin-1 isoforms, neuregulin EGF domain alone, neuregulin mutants, biologically active analogs of neuregulin, and any kind of neuregulin-like gene products that also activate the above receptors. Neuregulin also comprises neuregulin-1 (NRG-1), neuregulin-2 (NRG-2), neuregulin-3 (NRG-3), and neuregulin-4 (NRG-4). Neuregulin is also known as heregulin, neu differentiation factor, glial growth factor, acetylcholine receptor-inducing activity, and sensory and motor neuron-derived factor. Neuregulin also comprises variants with conservative amino acid substitutions that do not substantially alter their biological activity. Suitable conservative substitutions of amino acids are known to those of skill in this art and may be made generally without altering the biological activity of the resulting molecule. Those of skill in this art can recognize that, in general, single amino acid substitutions in non-essential regions of a polypeptide do not substantially alter biological activity.

The terms “functional” or “bioactive,” as used interchangeably herein, refer to mean a NRG-derived peptide having a non-amino acid chemical structure that mimics the structure of NRG or a NRG-derived peptide and retains the bioactivity and function of NRG in cellular and animal models. The function may comprise an improved desired activity or a decreased undesirable activity. Such a mimetic generally is characterized as exhibiting similar physical characteristics such as size, charge or hydrophobicity in the same spatial arrangement found in NRG or the NRG-derived peptide counterpart. A specific example of a peptide mimetic is a compound in which the amide bond between one or more of the amino acids is replaced, for example, by a carbon-carbon bond or other bond well known in the art (see, for example, Sawyer, Peptide Based Drug Design, ACS, Washington (1995), which is incorporated herein by reference). Non-limiting tests for a functional NRG are disclosed below. The peptides of the present invention are intended to be functional in at least one bioactivity assay. Tests for functionality are described below.

As used herein, “a” or “an” means “at least one” or “one or more.” The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

As used herein, “vector (or plasmid)” refers to discrete elements that are used to introduce heterologous deoxyribonucleic acid (DNA) into cells for either expression or replication thereof. Selection and use of such vehicles are well known within the skill of the artisan. An expression vector comprises vectors capable of expressing DNAs that are operatively linked with regulatory sequences, such as promoter regions, that are capable of effecting expression of such DNA fragments. Thus, an expression vector refers to a recombinant DNA or ribonucleic acid (RNA) construct, such as a plasmid, a phage, recombinant virus or other vector that, upon introduction into an appropriate host cell, results in expression of the cloned DNA. Appropriate expression vectors are well known to those of skill in the art and comprise those that are replicable in eukaryotic cells and/or prokaryotic cells and those that remain episomal or those which integrate into the host cell genome.

As used herein, “a promoter region or promoter element” refers to a segment of DNA or RNA that controls transcription of the DNA or RNA to which it is operatively linked. The promoter region comprises specific sequences that are sufficient for RNA polymerase recognition, binding and transcription initiation. This portion of the promoter region is referred to as the promoter. In addition, the promoter region comprises sequences that modulate this recognition, binding and transcription initiation activity of RNA polymerase. These sequences may be cis acting or may be responsive to trans acting factors. Promoters, depending upon the nature of the regulation, may be constitutive or regulated. Exemplary promoters contemplated for use in prokaryotes comprise the bacteriophage T7 and T3 promoters, and the like.

As used herein, “operatively linked” or “operationally associated” refers to the functional relationship of DNA with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it may be necessary to remove, add or alter 5′ untranslated portions of the clones to eliminate extra, potential inappropriate alternative translation initiation (i.e., start) codons or other sequences that may interfere with or reduce expression, either at the level of transcription or translation. Alternatively, consensus ribosome binding sites (see, e.g., Kozak, J. Biol. Chem., 266:19867-19870 (1991)) can be inserted immediately 5′ of the start codon and may enhance expression. The desirability of (or need for) such modification may be empirically determined.

The term “subject” refers to any living organism in which an immune response can be elicited. The term subject comprises, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered. In preferred embodiments, the subject is a mammal, including humans and non-human mammals. In a more preferred embodiment, the subject is a mammal. In the most preferred embodiment, the subject is a human.

The invention is described in more detail in the following subsections:

### I. Risk Factors for Brain Damage in Pre-Term Infants

An improved understanding of this maturity related risk for infants can be of major importance for those who wish to prevent neonatal brain damage and its long-term adverse developmental consequences. One traditional explanation is that preterm infants are (endogenously) more vulnerable to exogenous insults. Therefore, it was thought that improved neonatal care should lead to reduced white matter damage (WMD) risk and improved outcome. However, recent observational studies indicate that this hypothesis may not be true.

Another explanation for increased vulnerability of preterm infants is that they are more likely than their fullterm peers to experience exogenous insults. For example, intrauterine infection is both a risk factor for perinatal brain damage and is observed more frequently in the placentas of preterm infants than fullterm infants. Thus, the increased WMD risk of preterm infants can reflect their higher likelihood of being exposed to adversity.

The invention is based, in part, on the discovery that neuregulin can be an endogenous perinatal neuronal protector. NRG plays a role in neuronal development and is shown to be protective against potentially damaging mechanisms, such as mechanisms that lead to neuronal cell damage and/or death. These mechanisms comprise, but are not limited to, infection/inflammation, hypoxia-ischemia, and excitotoxicity. In addition, NRG can also be used to prevent, and or treat neuropsychiatric disorders, such as schizophrenia where patients exhibit altered levels of neuregulin function and display hypofunction of neuronal synapses.

In some embodiments, additional endogenous protectors can be co-administered with NRG to reduce the risk of prenatal brain damage. Low gestational age is a surrogate not only for increased vulnerability and adverse exposures, but also for many other endogenous and exogenous factors. Among these are substances that can be called ‘endogenous protectors’. Substances that qualify as endogenous protectors (a) can be endogenously available to the organism, (b) can be protective in experimental models of disease, (c) can offer biologically plausible interactions with presumed pathways of damage, and (d) can be associated with a decreased risk for perinatal brain damage in humans. Non-limiting examples of endogenous protectors comprise glucocorticoid and thyroid hormones.

Glucocorticoids are catabolic steroids that break down stored resources (fats, sugars and proteins) so that they can be used as fuels in times of stress. Glucocorticoids are characterized by the ability to bind with the cortisol receptor and trigger similar effects, including anti-inflammatory and immunosuppressive properties. They can even promote cell differentiation of specific cells at specific time points in development. Non-limiting example of glucocorticoids useful with this invention comprise dexamethasone, hydrocortisone, alclometasone, amcinonide, beclometasone, betamethasone, budesonide, ciclesonide, clobetasol, clobetasone, clocortolone, cloprednol, cortivazol, deflazacort, deoxycorticosterone, desonide, desoximetasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, rimexolone, tixocortol, triamcinolone, ulobetasol.

Hypothyroidism can adversely affect the neurological development and must be treated as soon as it is diagnosed. Preterm infants are prone to develop various degrees of deficiency. The thyroid produces two major hormones which regulate metabolism. Thyrotropin releasing hormone (TRH) made by the hypothalamus stimulates the release of thyroid stimulating hormone (TSH) from the anterior pituitary which regulates the production and release of thyroid hormones. There are several brand names of thyroid hormone available that contain the same synthetic thyroxine (T4).

### II. Neuregulin (NRG)

Neuregulins are a family of multipotent growth factors that comprise acetylcholine receptor inducing activities (ARIAs), growth factors, heregulins, and neu differentiation factors. Sequence information of human NRG-1 can be found, for example, under Accession No. Q02297, NCBI Accession No: AAA58639.1 (SEQ ID: 1). Various isomers can be found, for example, under Accession Nos. Q02297-1, (Alpha) (SEQ ID: 2), Q02297-2 (Alpha1A) (SEQ ID: 3), Q02297-3 (Alpha2B) (SEQ ID: 4), Q02297-4 (Alpha3) (SEQ ID: 5), Q02297-6 (Beta1, Beta1A) (SEQ ID: 6), Q02297-7 (Beta2) (SEQ ID: 7), Q02297-8 (Beta3, GGFHFB1) (SEQ ID: 8), Q02297-9 (GGF2, GGFHPP2) (SEQ ID: 9), Q15491-1 (SMDF) (SEQ ID: 10).

Neuregulins are developmentally regulated growth factors. Functionally, they play a critical role in the developing heart, nervous, and mammary systems. For example, neuregulin-1β was found to be secreted by the fetal lung fibroblast and stimulated control of fetal lung maturation through mesenchymal-epithelial interactions in a paracrine mechanism similar to the mechanism described for the developing heart, brain, and mammary systems.

The effects of neuregulins can be mediated through an interaction with ErbB receptors, a class of tyrosine kinase receptors related to the epidermal growth factor receptor. Neuregulins stimulate the tyrosine phosphorylation of these receptors and the subsequent activation of various signal transduction mechanisms. Neuregulins are synthesized as transmembrane precursors consisting of either an immunoglobulin-like or cysteine-rich domain, and EGF-like domain, a transmembrane domain, and a cytoplastic tail. The EGF-like domain of NRG-1 appears to be sufficient for activation.

Neuregulin-1 (NRG-1) was originally identified as a 44-kD glycoprotein that interacts with the Neu/ErbB2 receptor tyrosine kinase to increase its phosphorylation on tyrosine residues. The receptors for all NRG-1 isoforms are the ErbB family of tyrosine kinase transmembrane receptors. Through interaction with ErbB receptors, NRG-1 isoforms induce the growth and differentiation of epithelial, neuronal, glial, and other types of cells.

NRG-1 is the best-studied member and the prototype of a family of polypeptide growth factors encoded by four distinct genes, i.e., NRG-1, NRG-2, NRG-3, and NRG-4. NRG-1 binds and activates Erb3 and Erb4. Sequence information of NRG-2 can be found, for example, under Accession Nos. O14511 (SEQ ID: 11) and Q3MI86 (SEQ ID: 12). Sequence information of NRG-3 can be found, for example, under Accession Nos. P56975 (SEQ ID: 13), Q5VYH3 (SEQ ID: 14), Q6ZWG6 (SEQ ID: 15). Sequence information of NRG-4 can be found, for example, under Accession Nos. Q0P6N4 (SEQ ID: 16).

All 16 known isoforms of NRG-1 are encoded by the same gene and result from alternative splicing and usage of different promoters. A classification system (type I-III) is based on the type of epidermal growth factor (EGF)-like domain, N-terminal sequence, and initial synthesis as transmembrane or non-membrane protein. Type I NRG-1, also known as heregulin or NDF, contains an N-terminal immunoglobulin (Ig)-like domain prior to the EGF-like domain, which is followed by a specific hydrophobic stretch and a unique C-terminal domain. The EGF-like domain in type I NRG-1 can be either the alpha or beta variant, depending on whether the transcript comprises exon 7 or 8, respectively. Type II NRG-1, or glial growth factor-2 (GGF2), contains an N-terminal signal peptide, a Kringle-like domain, and an Ig-like domain prior to the EGF-like domain (beta variant). Type III NRG-1, or sensory and motor neuron-derived factor (SMDF), contains a unique N terminus with a hydrophobic stretch prior to the EGF-like domain (beta variant). Three additional types (IV-VI) are theoretically possible.

Single nucleotide polymorphisms (SNPs) are a genetic variation occurring when a single nucleotide differs, between members of the same species or between paired chromosomes in the same organism. Within a population, SNPs are typically assigned the minor or lower frequency allele at a genetic locus. When SNPs fall within the coding region of the gene, SNPs can result in the substitution of an amino acid in the protein sequence. In other instances, some SNPs can result in the termination of a protein sequence or a silent mutation with no change in protein sequence. On the other hand, the functional consequences of SNPs outside the coding region is less certain. In some cases, SNPs have been identified in regions upstream of the coding sequences, a potential promoter region, and have affected gene expression.

Several dozen SNPs have been identified in the neuregulin genetic locus. In many instances, SNPs within neuregulin have been associated with schizophrenia and potentially Alzheimer's disease, however the functions of most SNPs remain unknown since the majority of the SNPs are in noncoding regions of the locus. Common SNP sites within the neuregulin gene locus can comprise but are not limited to: SNP8NRG221132 (single nucleotide polymorphism “R”) (SEQ ID: 17), SNP8NRG221533 (single nucleotide polymorphism “Y”) (SEQ ID: 18), SNP8NRG241930 (single nucleotide polymorphism “K”) (SEQ ID: 19), SNP8NRG243177 (single nucleotide polymorphism “Y”) (SEQ ID: 20), and SNP8NRG433E1006 (nucleotide polymorphisms “R” and “Y”) (SEQ ID: 21).

### III. ErbB Signaling Pathways

NRG-1 and its ErbB tyrosine kinase receptors (ErbB 1-4) are highly expressed in the adult rodent and human brain. However, their functions are not well known. NRG-1 is expressed in CA3 pyramidal neurons that project to CA1, accumulates at various central synapses including the hippocampal CA1 molecular layer, and is processed and released at synapses in an activity dependent manner. In the adult brain, ErbB and NMDA receptors co-localize at glutamatergic postsynaptic sites and interact with PDZ (postsynaptic density 95/Discs large/zona occludens-1)-domain scaffolding proteins.

The ErbB receptors are a family of four transmembrane receptors, that bind multiple growth factors, including EGF, transforming growth factor-α, and NRG-1-4, among others. ErbB1, also known as the EGF receptor, does not bind NRG-1, while ErbB3 and ErbB4 do serve as its receptors. Historically, interest in ErbB receptors is rooted in their role as part of the erythroblastosis virus oncogene (v-Erb), which bears two domains in chickens, v-ErbA and v-ErbB. One line of interest in NRGs and ErbB receptors is in the fields of cancer biology and therapy since disarrangement of this signaling network plays a major role in the loss of normal growth arrest and dedifferentiation seen in cancer biology. Endogenous negative regulation of ErbB-receptor signaling might be of benefit in cancer therapy.

NRG and its related ErbB receptors influence the growth and maturation of immature oligodendrocytes, and maturation is disordered when a full complement of protein and receptor are not available. Damage to, or aberrant maturation of, developing oligodendrocytes can be a pathogenetic factor in diffuse perinatal WMD. In one embodiment of the invention, a method is disclosed to prevent WMD and its consequences comprising delivering NRG to the developing neuronal cells.

NRG can be coupled to a carrier to direct delivery to the tissue to be targeted (e.g., brain, lung). In some embodiments, the active agent, NRG, must traverse the blood-brain barrier. A hydrogen bonding unit can be conjugated to the NRG, such as to create an increased affinity with a receptor. For example, an Ant or Tat peptide can be a NRG conjugate.

### IV. Perinatal Brain Injury

Perinatal brain injury, especially in the preterm neonate, is likely to have multiple causes, such as exposure to infection/inflammation, hypoxia-ischemia, and excitotoxicity. For example, an prenatal infection can elicit a maternal/fetal inflammatory response, which either directly damages the developing brain (i.e., via cytokine-induced damage and/or leading to damage by sensitizing the developing brain to subsequent hits, such as a second inflammatory challenge, hypoxia-ischemia, or free radical attack.). In one aspect, the invention provides a method of reducing risk of perinatal brain damage through the administration and/or increased production of NRG. In one embodiment, NRG can reduce inflammation and/or oxidation-induced brain damage.

The invention is based on the discovery that NRG signaling can reduce perinatal brain damage. NRG-ErbB-signaling system intersects with inflammatory mechanisms. In addition, differential ErbB-heterodimerization patterns were observed in lung type-II cells in pro-inflammatory versus anti-inflammatory contexts. In the brain, the neuroprotective effect of NRG exposure prior to middle cerebral artery occlusion is accompanied by a prominent reduction in microglia activation and interleukin-1 mRNA expression in the penumbra, indicating a downregulation of peri-infarct inflammation by NRG. In one aspect, the invention provides a method of reducing inflammation and/or oxidation in the brain. NRG has anti-inflammatory and anti-oxidative properties in the brain. Recombinant human NRG can attenuate the production of super-oxide and nitrite by stimulating N9 microglial cells.

Nuclear factor KB (NF-κB) is a nuclear component of the cell's inflammatory response. NF-κB-inducing kinase (NIK) is one component of the signaling cascade initiated by pro-inflammatory cytokines such as tumor necrosis factor-α, lymphotoxin-β, and interleukin-1, but is only required for lymphotoxin-β signaling. NIK is recruited to all four ErbB-receptors and can activate NF-κB in wild-type, but not in NIK-deficient cells. On the other hand, NF-κB is not the predominant signal in white matter inflammation induced by lipopolysaccharide.

Three days after permanent middle cerebral artery occlusion (MCAO), neuregulin is found to be prominently expressed in neurons of the penumbra. In adult rat brain, ErbB4 (but not ErbB2 and ErbB3) protein is upregulated in neurons and macrophages/microglia in ischemic areas after MCAO. A single intravascular injection of NRG-1β (approximately 2.5 ng/kg) can reduce cortical infarct volume by >98% when given immediately before MCAO. Thus, the induction of ErbB receptors can be an adaptive response to prevent neuronal injury. The invention discloses that brain damage can be reduced and/or ameliorated in the developing brain following administration of NRG.

Some perinatal brain damage can be attributed to excitotoxicity. In models of excitotoxic brain damage, ibotenate injection leads to neuronal loss and porencephalic cysts by acting as a glutamate analogue on the NMDA receptor. In cerebellar granule cells from 9-day-old mice, NRG upregulates the expression of the NR2C-subunit of NMDA receptors, which appears to be associated with an increased resistance of neurons to the adverse effects of neurotoxicity.

At least part of NRG's purported role in the etiology of schizophrenia might be due to a link between NRG/ErbB and glutamate/NMDA signaling. In addition, NRG-1β appears to play a glutamate-dependent role in memory. Rats, when exposed to 5 weeks of learning to navigate a maze, show increased brain expression of NRG-1β, but not if they also receive the glutamate blocker MK-801.

In one aspect, the invention provides a method of reducing brain damage in neonates through the stabilization of ErbB-receptor expression. The inventions shows that the NRG/ErbB signaling is protective during brain development. In some embodiments, the invention provides for antenatal administration of NRG. In other embodiments, NRG can be co-administrated with other endogenous protector, such as glucocorticoids, to reduce the incidence of neonatal brain damage.

In another aspect, a prophylactic method can be provided to protect the subject's brain against perinatal inflammatory, hypoxic-ischemic, and/or excitotoxic insults through direct NRG administration. NRG can enter the brain after intravenous, intra-arterial, intraperitoneal and intraamniotic administration. In experimental autoimmune encephalitis (one laboratory model for multiple sclerosis), systemic treatment with NRG reduces demyelination and enhances remyelination. This effect might not be due to NRG acting directly on oligodendroglia, but via induction of an environment more favorable to remyelination, possibly through modulation of the immune response. Such an environment can help the perinatal brain survive preterm birth without sustaining long-term damage.

In one aspect, the invention provides a method of administration of NRG for exogenous indirect or direct neuroprotective intervention. NRG and its receptors play key roles in brain development and damage. The invention recognizes that regulation of the NRG/ErbB system can modulate perinatal brain development. In addition, the importance of the NRG/ErbB system in the perinatal setting is supported by the findings that there is enhanced fetal lung surfactant synthesis by NRG-1β, and that all 4 ErbB receptors are present at the fetal endothelium as early as 24 weeks gestation. Thus, NRG can act as an endogenous protector in the perinatal brain and other organs (e.g. lung, eye, and gut).

### V. Evaluating NRG Expression and Activation of ErbB Receptors

Several NRG isoforms exist, all produced by alternative splicing of the same region on human chromosome 8p22 and all containing an EGF-like domain. For example, one of these isoforms, NRG-1β, appears to help signal the onset of surfactant synthesis in the fetal lung and can provide a protective function in experimental brain ischemia and several adult neurologic diseases. In addition, administration of NRG-1 has been shown to reduce ischemic cortical damage following transient middle cerebral artery occlusion in adult rats. Thus one aspect of the disclosed invention is directed to determining the expression level of NRG isoforms available in the cells of interest, for example NRG-1β.

Methods of assessing expression levels of NRG in a subject are known by those skilled in the art. Expressed NRG or fragments or derivatives thereof can be isolated and purified from a sample from a subject by known methods. The sample can be cells of interest or cellfree material (e.g. supernatant of cells, urine, liquor, or amniotic fluid) that can comprise but are not limited to fetal cells, umbilical cord cells, neuronal cells, blood cells, lung cells, endothelial cells and any other NRG expressing cell type. In addition, the samples can be obtained by those experienced in the art, including but not limited to, biopsying a fetus, obtaining fetal cells while in a pregnant subject, biopsying a neonate, flushing umbilical cord endothelial cells, chorionic villi sampling or cell harvest from the amionic cavity. Nonlimiting examples of methods to assess expression levels quantitatively or qualitatively can comprise, quantitative real time polymerase chain reaction (qRT-PCR), northern blotting, western blotting, enzyme-linked immunosorbent assay (ELISA), confocal microscopy, immunohistochemistry, protein arrays and flow cytometry. Quantitative or non quantitative PCR can also be used to amplify desired nucleic acid sequences of NRG from a genomic, cDNA or RNA library. Isolated oligonucleotide primers representing known NRG coding or noncoding sequences can be used as primers in the PCR. The synthetic oligonucleotides can be utilized as primers to amplify by PCR sequences from ribonucleic acid (RNA) (icon/nucleic acid (DNA), or more preferably a cDNA library, to analyze expression levels.

Genetic polymorphisms within the NRG genetic locus can also be associated with altered expression levels of NRG transcript or protein. For example both immature and mature cells with at least one SNP8NRG221533 allele, a single nucleotide polymorphism within the NRG genetic locus, have been shown to be capable of producing variant levels of NRG-1β depending on the nucleotide present in the polymorphism. In addition, schizophrenic samples have shown altered NRG-ErbB activation levels depending on the SNP present in NRG-1. Thus in one aspect of the invention, subjects can be screened for single nucleotide polymorphisms (SNPs) within the NRG gene locus and depending on the level of NRG expression a prediction can be made whether the subject is at risk for developing a neurological disorder. Methods of screening polymorphisms are well know in the art and can be used to determine the polymorphism genotype of the subject. Non-limiting examples can comprise: DNA or RNA sequence analysis, PCR, restriction fragment length polymorphisms, restriction site polymorphisms, protein sequence analysis, ELISA and any combination thereof. In another embodiment, subjects carrying predetermined polymorphisms (e.g., SNP8NRG221533) can be assessed for NRG gene expression levels. Neuregulin and the corresponding SNPs within the gene can be isolated and purified by known methods and expression levels of NRG in a subject can be determined using any of a variety of assays known to those skilled in the art.

In another embodiment, polymorphisms in the genetic locus encoding for ErbB receptor family members can influence the activation of neuregulin signaling pathways. ErbB receptor polymorphisms can also result in abnormally low levels of activation of neuregulin signaling and can lead to perinatal morbidity despite normal expression levels of NRG. Thus, screening for SNPs in the ErbB receptor genetic locus can also be a predictor for infants prone to long term disability. Methods similar to those used to assess expression levels of NRG can also be used to measure ErbB receptor expression levels. In addition, methods to evaluate activation of ErbB receptors in response to NRG are also well known in the art and can include, but are not limited to: quantitative real time polymerase chain reaction (qRT-PCR), Southern blotting, northern blotting, western blotting, enzyme-linked immunosorbent assay (ELISA), confocal microscopy, immunohistochemistry, protein arrays and flow cytometry.

Levels of neuregulin expression and pathway activation in a subject can be compared to one or more other subjects with a similar age range, gender, genotype, species etc. or can be compared to one or more other subjects of a different age, gender, race, genotype, species etc. to determine qualitative expression differences. In addition, levels of neuregulin expression and activation of ErbB receptors can be measured over a time range for a subject to determine fluctuations of expression and activation over extended time periods. The time range can be measured in days, weeks, months or years. In one embodiment, neuregulin expression levels can be measured by assessing transcript levels in a subject. In another embodiment, neuregulin protein levels can be measured. In yet another embodiment, neuregulin activation of ErbB receptors can be measured. Additionally, polymorphisms can be compared in a subject and neuregulin levels can be compared between one or more other subjects displaying the same polymorphisms or lacking the polymorphisms.

In one aspect of the invention, subjects with reduced levels or deficient in neuregulin can be supplemented with neuregulin. In another embodiment, subjects with reduced levels or deficient in activation of neuregulin associated signaling pathways can be supplemented with neuregulin. In preferred embodiments, subjects with reduced levels of neuregulin expression can be supplemented with neuregulin to reduce or protect against disorders associated with neuronal cell damage. Methods for treating neuregulin deficiency or deficit are further delineated in Section VII.

### VI. Screening Assays for Biologically Active NRG

The ability of NRG to produce a neuroprotective effect in a subject can be determined using any of a variety of known assays. For example, the ability of NRG to prevent cell damage, death, and/or function after an injury, e.g., an hypoxic/ischemic injury, can be determined histologically (e.g., by assaying tissue loss, immature oligodendrocyte loss, or myelin basic protein (MBP) expression as set forth in the examples below). In addition, binding assays can be used to determine the affinity between NRG and ErbB receptors. For example, kinase assays can be used to determine the binding and activation of ErbB3 or ErbB4 protein kinases when exposed to NRG. In addition, an experimental model for inflammatory processes, like a lipopolysaccharide (LPS) challenge of different type of cells, e.g., 3T3 fibroblasts and neural cells, as well as in vivo animal models (intra tecthal, intraperitoneal, intravasal, and intraamniotic injection) can be used to determine if NRG has a cytoprotective effect. This effect can be accompanied by corresponding changes of apoptotic markers and inflammatory markers, monitored by staining western blots with antibodies against PARP-1 and Cox-2.

Other tests that can be used to determine the ability of NRG to produce a neuroprotective effect in a subject comprise standard tests of neurological function in human subjects or in animal models of brain injury such as memory tests (e.g., Morris water maze, T maze, spontaneous alternation test, and bar-pressing task); locomotor activity (e.g., vertical movements, sniffing, grooming, coordination, and spontaneous locomotor activity); exploratory activity (e.g., novel large cage test); anxiety (e.g., freezing test, hole-board test, elevated plus maze, forced swimming test); nociception (e.g., hot plate test); feeding motivation; and aggressive behavior. Examples of such tests can be found in, for example, Miyachi et al. (1994) Neurosci. Lett. 175:92-94; Vaillend et al. (1995) Behay. Genet. 25:569-579; Fiore et al. (1996) Exp. Parasitol. 83:46-54; Valentinuzzi et al. (1998) Learning & Memory 5:391-403; Andreatini et al. (1999) Braz. J. Med. Biol. Res. 32:1121-1126; Crabbe et al. (1999) Science 284:1670-1672; Rao et al. (1999) Psychopharm. 144:61-66; and U.S. Pat. No. 5,447,939 (1995).

### VII. Pharmaceutical Compositions

The invention contemplates delivering the therapeutic agent, i.e., NRG, to the pregnant mother so as to indirectly deliver it to the fetus. In other embodiments, the therapeutic agent can be administered directly to the fetus, or to the neonate. The pharmaceutical composition used can be chosen based on whether direct or indirect delivery is selected.

As described in detail below, the pharmaceutical compositions comprising a composition of the invention (e.g., NRG) can be formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream, foam, or suppository; or (5) aerosol, for example, as an aqueous aerosol, liposomal preparation or solid particles containing the compound.

Pharmaceutical compositions and packaged formulations comprising a composition of the invention (e.g., NRG) and a pharmaceutically acceptable carrier are also provided by the invention. In the method of the invention, NRG can be administered in a pharmaceutically acceptable formulation. Such pharmaceutically acceptable formulation typically comprises NRG as well as a pharmaceutically acceptable carriers) and/or excipient(s). As used herein, “pharmaceutically acceptable carrier” comprises any and all solvents, dispersion media, coatings, antibacterial and anti fungal agents, isotonic and absorption delaying agents, and the like that are physiologically compatible. Excipients comprise pharmaceutically acceptable stabilizers and disintegrants. The present invention pertains to any pharmaceutically acceptable formulations, comprising synthetic or natural polymers in the form of macromolecular complexes, nanocapsules, microspheres, or beads, and lipid-based formulations including oil-in-water emulsions, micelles, mixed micelles, synthetic membrane vesicles, and resealed erythrocytes. Supplementary active compounds can also be incorporated into the compositions.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the antimicrobial agents or compounds of the invention from one organ, or portion of the body, to another organ, or portion of the body without affecting its biological effect. Each carrier should be “acceptable” in the sense of being compatible with the other ingredients of the composition and not injurious to the subject. Some examples of materials which can serve as pharmaceutically acceptable carriers comprise: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid, and the like. It can also be desirable to comprise isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

A pharmaceutical composition of the invention can be formulated to be compatible with its intended route of administration. Examples of routes of administration comprise parenteral, intravenous, intradermal, subcutaneous, oral (e.g., inhalation), transdermal (e.g., topical), transmucosal, and rectal administration. Preferably, the route of administration can be oral. Solutions or suspensions used for parenteral, intradermal, or subcutaneous application can comprise the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use comprise sterile aqueous solutions (where water soluble) or dispersions and sterile powders for the extemporaneous preparation of sterile injectable solutions or dispersion. For intravenous administration, suitable carriers comprise physiological saline, bacteriostatic water, Cremophor EL™ (BASF, Parsippany, N.J.) or phosphate buffered saline (PBS). In all cases, the composition must be sterile. The composition can also be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to comprise isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NRG) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation can be vacuum drying and freeze-drying which yields a powder of the active ingredient plus any additional desired ingredient from a previously sterile-filtered solution thereof.

Oral compositions generally comprise an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier can be applied orally and swished and expectorated or swallowed. Pharmaceutically compatible binding agents, and/or adjuvant materials can be comprised as part of the composition. The tablets, pills, capsules, troches and the like can contain any of the following ingredients, or compounds of a similar nature: a binder such as microcrystalline cellulose, gum tragacanth or gelatin; an excipient such as starch orlactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated can be used in the formulation. Such penetrants are generally known in the art, and for transmucosal administration comprise, for example, detergents, bile salts, and fusidic acid derivatives. Transmucosal administration can be accomplished through the use of nasal sprays or suppositories. For transdermal administration, the active compounds can be formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that can protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It can be especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical formulation, used in the method of the invention, contains a therapeutically effective amount of the NRG. A “therapeutically effective amount” refers to an amount effective, at dosages and for periods of time necessary, to achieve the desired result. A therapeutically effective amount of the NRG can vary according to factors such as the disease state, age, and weight of the subject, and the ability of NRG (alone or in combination with one or more other agents) to elicit a desired response in the subject. Dosage regimens can be adjusted to provide the optimum therapeutic response. A therapeutically effective amount can also be one in which any toxic or detrimental effects of the NRG are outweighed by the therapeutically beneficial effects. A non-limiting dosage range (i.e., an effective dosage) can be from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

For humans, the active dose of NRG to protect against brain damage in neonates can generally be in the range of from about 0.001 mg to about 200 mg per kg. However, it is evident to one of skill in the art that dosages can be determined by the attending physician, according to the method of administration, patient's age, weight, counter indications and the like. Administration of therapeutically effective amounts of NRG as used herein can encompass oral, parenteral, intravenous, intramuscular, sub-cutaneous, transdermal, intratechal, rectal and intra-nasal administration.

Administration of cells and other treatments can be carried out by various methods, and the methods need not be the same for each component. Generally, the molecule can be administered by any known route of administration, including intravenously, orally, or intracerebrally (e.g., intraventricularly, intrathecally, or intracisternally, or directly into the brain). The dose can vary depending on the method of administration. Doses determined in rats are typically scaled up for human treatments. The scaling to be used depends upon the method of delivery. If the molecule is to be delivered systemically (e.g. orally or intravenously) then the scaling is by body weight, where a typical rat weighs 300 grams and a typical human weighs 70 kg. If the compound is to be delivered to the cerebrospinal fluid (e.g. intracisternal, intraventricular), scaling is by brain surface area. A typical rat brain has a surface area of 1 cm², and a typical human brain has a surface area of 1000-10,000 cm², depending upon whether all of the various folds buried in convolutions are counted or not. If the compound is to be delivered to the brain tissue, scaling is done by brain mass. A typical rat has a 2 g brain, while the typical human brain is 2 kg. Thus, if a single treatment of 0.5 μg given intracisternally is effective in a rat, an intracisternal injection of 0.5 mg would be effective in a human patient. Of course exact dosages can be adjusted according to the weight of the patient and other criteria. It is anticipated that effective dosage for all three general routes of administration can range from 0.001-1000 mg total for administration to spinal fluid or brain tissue. In preferred embodiments, the dosage can be in a range from 0.001-200 mg, 0.01-100 mg, 0.1-10 mg or 0.5-5 mg.

Compounds can be administered in a single dose or they can be distributed in a series of smaller doses. For example, intracisternal administration can consist of a single injection given, for example, six hours after an injury, a pair of injections, given, for example, 24 and 48 hours after an injury, or, if necessary, a series of injections of, for example, 0.1 mg/injection, or a 1 mg injection, given biweekly (for example, every 34 days) in a treatment regimen that occurs at least six hours following the ischemic episode. The treatment regimen can last a number of weeks. For example, treatment can be given to a pregnant female at the beginning of pregnancy and continued throughout the gestation period.

It is to be noted that dosage values can vary with the severity of the condition to be alleviated. It is to be further understood that for any particular subject, specific dosage regimens can be adjusted over time according to the individual need and the professional judgment of the person administering or supervising the administration of NRG and that dosage ranges set forth herein are exemplary only and are not intended to limit the scope or practice of the claimed invention.

The invention, in another embodiment, provides a pharmaceutical composition consisting essentially of NRG and a pharmaceutically acceptable carrier, as well as methods of use thereof to modulate disorders associated with neuronal cell death e.g., PVL, mental retardation, neonatal stroke, cerebral palsy, fetal gray matter injury, and/or fetal white matter injury with the composition. In one embodiment, the pharmaceutical composition of the invention can be provided as a packaged formulation. The packaged formulation can comprise a pharmaceutical composition of the invention in a container and printed instructions for administration of the composition for treating a subject having a disorder associated with neuronal cell damage. The instructions can comprise directions for the treatment of adults and/or children. Preferably, the instructions comprise directions for the treatment of neonates. In another preferred embodiment, the instructions comprise directions for the treatment of a fetus via the administration of the composition to the pregnant mother. The pharmaceutical compositions can be comprised in a container, pack, or dispenser together with instructions for administration.

One skilled in the art will appreciate further features and advantages of the invention based on the above-described embodiments. Accordingly, the invention is not to be limited by what has been particularly shown and described, nor by the examples set forth below, except as indicated by the appended claims. All publications and references cited herein are expressly incorporated herein by reference in their entirety.

### EXAMPLES

This invention is further illustrated by the following examples which should not be construed as limiting. The following experiments were performed to demonstrate various aspects of the invention.

### Example 1

### Materials

Human NRG-1β1 (amino acids 176-246) and NRG-1α2 (amino acids 177-241) epidermal growth factor (EGF)-like domain peptides (R & D Systems, Minneapolis, MC), and recombinant human NRG-1 protein (GenWay Biotech, Inc., San Diego, Calif.) can be stabilized in 0.1% BSA. NRG-1b was expressed and purified as previously described (Carraway K L, et al. Nature 1997, 387: 512-516, and Crovello C S, et al. J Biol Chem 1998, 273:26954-26961).

### Example 2

### Neuroprotective Effect of NRG in Cerebral Ischemia

LPS challenge of 3T3 fibroblasts, A 549 cells, V56 embryonic stem cells and neurally differentiated V56 embryonic stem cells were performed by exposing cells to 100 ng/ml lipopolysaccharide (LPS, E. coli 0111:B4 LPS from Sigma) for 60 min in the presence or absence of NRG and related compounds. Cell pellets were further investigated by Western blot staining with anti Cox-2 and anti iNOS antibodies of 1D polyacrylamide gels.

Three different functional models: induction of chemical ischemia, induction of excitotoxic cell death by 100 μM NMDA (or 100 μM HCA) and induction of neuronal death by 10 μM α-amyloidl-40 (Bachem, Germany). All three challenges induced an initial calcium overload, which obviously initiated proapoptotic and proinflammatory events, leading eventually to neuronal dysfunction and cell death. Western blots of cellular fractions with or without NRG application were evaluated and showed increased levels of apoptotic marker PARP-1 and inflammatory marker Cox-2.

### Example 3

### In Vitro Testing of NRG and Neuroprotective Effects

Endothelial cells were harvested from human umbilical cord vessels within 1-5 days of delivery. Briefly, umbilical veins and arteries were cannulated with blunt needles, rinsed with sterile PBS buffer, and type I collagenase (0.04%, GIBCO, Invitrogen) was installed for 25 minutes at 37° C. Cell-collagenase suspension were collected and centrifuged at 250×g for 5 minutes at 4° C. Cells were resuspended, and plated in endothelial cell growth medium containing 10% fetal calf serum. Cells were washed after 2-3 hours of adherence, new medium was added, and cells were grown to confluence. Umbilical cords with histologic evidence of inflammation (funisitis) were excluded to reduce the likelihood of obtaining biased results from activated endothelial cells. Umbilical cords were collected from babies born either earlier than 30 weeks gestation (preterm group) or after completion of the 37th week of gestation (mature group). We used cells up to the 5th passage for the experimental assays. Before treatment, cells were starved for two hours in Dulbecco's Modified Eagle's medium (DMEM) containing 0.1% FCS. Treatment was performed with 100 ng/ml LPS. Cells were treated for 1.5, 3, 6, 12 and 24 hours. Untreated controls were kept in culture in DMEM for the same duration.

HUVEC were grown on glass slides for 24 hours and starved in serum-free DMEM for 2 hours. Cells were either treated with LPS (100 ng/ml) for 30 min. or 24 hours at 37° C. or left in DMEM as a control for the same duration. Immunofluorescence was performed as previously described (45). Briefly, cells were fixed with 3% paraformaldehyde for 20 minutes followed by permeabilisation with 0.2% Triton X14 100 for 2 minutes. After 1 hour blocking in 10% normal goat serum, fixed cells were incubated with the specific primary NRG antibody (rabbit polyclonal IgG Neuregulin-Ab 2; Neo Markers) and a specific erbB4 receptor antibody (mouse monoclonal IgG erbB4 (C7); Santa Cruz) at room temperature for 1-2 hours. Cells were washed with PBS and incubated with the appropriate secondary antibody conjugated with Alexa488 or Alexa568. Subsequently cells were incubated in 4′,6-Diamidino-2-phenylindole dihydrochloride (DAPI) (Sigma) for 10 minutes. Cells were mounted in Gelvatol/DABCO and analyzed using a Leica TCS-SP2 confocal laser scanning microscope.

Our first result was that both immature and mature human umbilical venous endothelial cells (HUVECs) expressed NRG-1β. In non-stimulated (but serum-starved) cells, NRG-1β was diffusely expressed in the cytoplasm in both mature and immature HUVECs. Mature cells also showed remarkable NRG-1β enrichment within the cell membrane after 30 min of both serum starvation and LPS-treatment. With increasing duration of exposure to LPS, NRG-1β seemed to enrich in the nuclear region. In immature HUVECs, NRG-1β localization was more diffuse in and around the nuclear region with little membrane staining in the untreated cells. The localization in the cell membrane fully disappeared after LPS stimulation. With increasing duration of LPS-exposure, NRG-1β localization increased in the nucleus in immature cells. LPS-treatment appeared to amplify this effect with special enhancement in the nucleoli in both gestational ages.

Twenty-four hours of LPS-treatment led to an increase in NRG-1β protein expression in mature cells. We found that treated cells raised their expression to a mean of 233±23% (mean±SEM, N=2) of control cells, whereas immature cells significantly (p=0.03) reduced the NRG-1β protein expression down to 79±8% (mean±SEM, N=3) of control cells. Each assay was carried out in quadruplicates. NRG-1β concentrations in corresponding supernatants were increased after LPS exposure over controls in mature (167±50%, mean±SEM, N=2) and immature cells (144±5%, mean±SEM, N=3). In summary, our results suggest that NRG-1β is expressed and might even be systemically released by human umbilical endothelial cells in response to LPS challenge.

### Example 4

### In Vivo Testing of NRG and Neuroprotective Effects

A bioactive NRG conjugated with a lipophilic moiety and identified as having an enhanced capability of entering neural cells in culture can be tested for functionality in vivo in an animal model (e.g., in a mammal, such as in mouse or rat). For example, the NRG can be administered to an animal, and the NRG evaluated with respect to its biodistribution, e.g., is uptake into neural cells, its stability, and its ability to stabilize expression of a target gene, ErbB, in the neural cells.

NRG can be injected directly into a target region of the brain (e.g., into the cortex, the substantia nigra, the globus pallidus, the hippocampus, or the striatum), and after a period of time, the brain can be harvested and tissue slices examined for distribution of the agent. In such experiments using concomitant ibotenate injection as an excitotoxic damage initiator, NRG appears to exert protective effects in both the grey and white matter of the murine brain.

NRG can also be evaluated for its intracellular distribution. The evaluation can comprise determining whether NRG was taken up into the cell. The evaluation can also comprise determining the stability (e.g., the half-life) of NRG. Evaluation of NRG in vivo can be facilitated by use of a NRG conjugated to a traceable marker (e.g., a fluorescent marker such as Cy3, Cy5, FITC, rhodamine, or fluorescein; a radioactive label, such as ³²P, ³³P, or ³H; gold particles; or antigen particles for immunohistochemistry).

NRG conjugated to a lipophilic moiety can be evaluated with respect to its ability to up regulate target gene expression, e.g., ErbB3 or ErbB4. Levels of target gene expression in vivo can be measured, for example, by in situ hybridization, or by the isolation of RNA from tissue prior to and following exposure to NRG. Target RNA can be detected by any desired method, including but not limited to RT-PCR, northern blot, or RNAase protection assay. Alternatively, or additionally, target gene expression can be monitored by performing western blot analysis on tissue extracts treated with a NRG.

NRG conjugated to a lipophilic agent for enhanced uptake into neural cells, and/or NRG in a pharmaceutically acceptable carrier can be tested in a mouse model for infection/inflammation, hypoxia-ischemia, and excitotoxicity, neuronal cell damage, and/or neurological disease. For example, NRG can be tested in a mouse model of brain injury such as memory tests (e.g., Morris water maze, T maze, spontaneous alternation test, and bar-pressing task); locomotor activity (e.g., vertical movements, sniffing, grooming, coordination, and spontaneous locomotor activity); exploratory activity (e.g., novel large cage test); anxiety (e.g., freezing test, hole-board test, elevated plus maze, forced swimming test); nociception (e.g., hot plate test); feeding motivation; and aggressive behavior. Examples of such tests can be found in, for example, Miyachi et al. (1994) Neurosci. Lett. 175:92-94; Vaillend et al. (1995) Behay. Genet. 25:569-579; Fiore et al. (1996) Exp. Parasitol. 83:46-54; Valentinuzzi et al. (1998) Learning & Memory 5:391-403; Andreatini et al. (1999) Braz. J. Med. Biol. Res. 32:1121-1126; Crabbe et al. (1999) Science 284:1670-1672; Rao et al. (1999) Psychopharm. 144:61-66; and U.S. Pat. No. 5,447,939 (1995).

### Example 5

### Assessing Incidence of Polymorphisms in NRG-1β

We evaluated 54 small for gestation age children <32 weeks gestation from the Developmental Follow Up Program at Hannover Medical School (Germany). Children were at least two years of age. A comprehensive physical examination and the Denver developmental screening (DDST) II test were administered by one paediatrician. Clinical data were abstracted from medical charts. A whole blood sample was obtained for genetic analysis. We established SNP8NRG221533 genotypes by polymerase chain reaction (PCR) and restriction fragment length polymorphism (RFLP) analysis.

Endothelial cells were harvested from human umbilical cord vessels within 1-5 days of delivery. Briefly, umbilical veins and arteries were cannulated with blunt needles, rinsed with sterile PBS buffer, and type I collagenase (0.04%, GIBCO, Invitrogen) was installed for 25 minutes at 37° C. Cell-collagenase suspension was collected and centrifuged at 250×g for 5 minutes at 4° C. Cells were resuspended, and plated in endothelial cell growth medium containing 10% fetal calf serum. Cells were washed after 2-3 hours of adherence, new medium was added, and cells were grown to confluence. Umbilical cords with histologic evidence of inflammation (funisitis) were excluded to reduce the likelihood of obtaining biased results from activated endothelial cells. Umbilical cords were collected from babies born either earlier than 30 weeks gestation (preterm group) or after completion of the 37th week of gestation (mature group). We used cells up to the 5th passage for the experimental assays. Before treatment, cells were starved for two hours in Dulbecco's Modified Eagle's medium (DMEM) containing 0.1% FCS. Treatment was performed with 100 ng/ml LPS. Cells were treated for 1.5, 3, 6, 12 and 24 hours. Untreated controls were kept in culture in DMEM for the same duration.

Human umbilical venous endothelial cells (HUVEC) were grown on glass slides for 24 hours and starved in serum-free DMEM for 2 hours. Cells were either treated with LPS (100 ng/ml) for 30 min. or 24 hours at 37° C. or left in DMEM as a control for the same duration. Immunofluorescence was performed. Cells were fixed with 3% paraformaldehyde for 20 minutes followed by permeabilisation with 0.2% Triton X14 100 for 2 minutes. After 1 hour blocking in 10% normal goat serum, fixed cells were incubated with the specific primary NRG antibody (rabbit polyclonal IgG Neuregulin-Ab 2; Neo Markers) and a specific erbB4 receptor antibody (mouse monoclonal IgG erbB4 (C7); Santa Cruz) at room temperature for 1-2 hours. Cells were washed with PBS and incubated with the appropriate secondary antibody conjugated with Alexa488 or Alexa568. Subsequently cells were incubated in 4′,6-Diamidino-2-phenylindole dihydrochloride (DAPI) (Sigma) for 10 minutes. Cells were mounted in Gelvatol/DABCO and analyzed using a Leica TCS-SP2 confocal laser scanning microscope.

We genotyped both whole blood samples from the children of the pilot study and HUVECs obtained from those umbilical cords used for RNA-extraction, ELISA and confocal microscopy. Genomic DNA was isolated after an overnight incubation of the cells with proteinase K (Merck) using standard phenol-chloroform extraction. Polymerase chain reactions (PCR) were performed in a 20 μl volume including 100 ng of genomic DNA, 4 μl Q solution (Quiagen), 2 μl 10×PCR buffer (Amersham), 2 μl of each primer (5 mM) (5′ Primer 1) 5′-ACCTAAGATGTCCAAGAGACAG-3 (forward) (SEQ ID: 22) and (3′ Primer 1) 5′-GACTGGAAGCCATGTATCTTTATTGT-3′ (reverse, both from Invitrogen) (SEQ ID: 23), 1.2 μl dNTPs (2 mM each, Amersham), 1.2 μl MgCl2 (25 μM, Quiagen), 6 μl H2O, and 0.1 μl HotStart Taq DNA polymerase (5 U/μl, Qiagen). Thirty-six cycles were performed with 15 min denaturation at 95° C., 1 min annealing at 62° and 1 min extension at 72° C. The artificially introduced mismatch in the reverse primer allowed for a subsequent allelic discrimination by the restriction enzyme RsaI. PCR products were incubated with RsaI (New England BioLabs) at 37° C. overnight and restriction fragment length analysis (RFLP) was performed by 3% agarose gel electrophoresis.

All sample measurements were carried out in quadruplets. Cells were scraped, lysed by sonication (3×30 sec), lyophilized, and resuspended in 200 μl PBS before they were applied to Elisa wells. The amount of total protein was measured by a highconcentration microplate assay using a commercially available reagents kit (DC Protein Assay, Bio-Rad) following the manufacturer's protocol.

The Elisa was performed using the Human NRG-1-beta 1/HRG-1-beta 1 DuoSet (DY377 R&D Systems). Ninety-six well-plates were coated overnight with 100 μl capture antibody per well (4 mg/ml Heregulin Ab-1 Clone 7D5, Neomarkers) at 4° C. After overnightblocking with 1% protease-free BSA and 5% sucrose, 100 μl of sample or standard were incubated overnight at 4° C. 100 μl detection antibody (200 ng/ml Heregulin/Neuregulin Ab-2, biotin-labeled, Neomarkers), solved in PBS with 1% protease-free BSA and 2% normal goat serum was added for two hours. Fifty microliters of Streptavidin HRP in 10 ml diluent were distributed to the wells (100 μl per well) and incubated for 20 minutes in the dark. One hundred microliters of substrate solution per well was incubated for 20 minutes in the dark. Substrate reaction was stopped by adding 50 μl 2N H2SO4. Photometric extinction was measured by 450 nm vs 570 nm. NRG-concentration was calculated based on 1000 μg of total protein and presented as % of controls.

RNA isolation was performed by using a guanidinium-based cell lysis and subsequent extraction method with acidic phenol solution (ABgene). RNA yield and quality were measured photometrically and by agarose gel electrophoresis. One microgram of total RNA was subjected to reverse transcription using the First-Strand cDNA synthesis kit (Amersham Biosciences). Real-time PCR was performed using a commercially available primer assay for NRG-1 and a SYBR Green Master Mix (Quiagen) following the manufacturer's protocol with 40 cycles and an annealing at 60° C. β-actin served as the housekeeping gene and was amplified on the same plate using primers (5′ Primer 2) 5′-AGATGACCCAGATCATGTTTGAG-3′ (SEQ ID: 24) and (3′ Primer 2) 5′-GAGTCCATCACGATGCCAGTG-3′ (SEQ ID: 25). We calculated means and DCt values using the ABI PRISM 7000 SDS software (Applied Biosystems) for relative quantification. Corresponding control values were subtracted from treated samples to calculate DDCt values. A negative result indicates a higher NRG-1 gene expression in the treated sample compared to the respective untreated control.

Forty-two (77%) of 54 infants in our study had at least one C-allele (CC: N=6; CT: N=36; TT: N=12) (Table 1). Ten (19%) of all children had cPVL as clinical outcome, 5 (9%) had cerebral palsy, and 9 (17%) had developmental delay. Comparing the 42 children homo- or heterozygous for the C-allele with those who had a TT-genotype, these percentages were 17% (CC/CT) vs. 25% (TT) for cPVL, 5% (CC/CT) vs. 25% (TT) for CP, and 18% (CC/CT) vs. 33% (TT) for developmental delay, respectively. Among those homozygous for the C-allele, all morbidity outcomes were absent. The one-sided Cochran-Armitage test for trend was p=[CD14]0.02 for cerebral palsy and p=0.03 for developmental delay even in this small sample, confirming a compromising reduced risk in infants with any C-allele.

Among the 54 children born before 32 weeks of gestation in our study, 63% (N=34) were born before 30 weeks of gestation, 44% (N=24) were less than 1000 g in birth weight, and 22% (N=12) were small for gestational age (SGA)), i.e. birth weight below 10th percentile (27). Infant's sex was equally distributed in both groups. There were no significant associations between perinatal risk factors and SNP8NRG221533 genotype. However, infants carrying at least one C-allele were appreciably less likely to be born after preterm labor (60%) than those with two T-alleles (83%), and also slightly less likely to be male (45% vs 67%).

Low gestational age and birthweight were the strongest risk factors for all three morbidity outcomes, cPVL, cerebral palsy and developmental delay. Among the other perinatal variables, only chorioamnionitis and preterm labor appeared to be potential confounders of the observed relationship between SNP8NRG221533 genotype and outcomes by virtue of being associated with both, genotype and outcomes.

Additional analyses revealed that adjustment for the single potential confounders gestation <30 weeks, birth weight <1000 g, male gender, preterm labour or chorioamnionitis did not result in a reduction of the effect of the SNP8NRG221533 genotype on outcome observed in univariable analyses. Of note, we considered our dataset too small to adjust for more than one variable at a time.

We found support for a dose-response-relationship between the amount of NRG-1β expression and (1) maturity level of cells and (2) the presence of the C-allele of SNP8NRG221533. In preterm infants, the presence of at least one C-allele was associated with a reduced risk for adverse neurologic outcome.

While the present invention has been described in terms of specific methods, structures, and devices it is understood that variations and modifications will occur to those skilled in the art upon consideration of the present invention. For example, the methods discussed herein can be utilized beyond reducing or assessing risk of neuronal cell damage in some embodiments. As well, the features illustrated or described in connection with one embodiment can be combined with the features of other embodiments. Such modifications and variations are intended to be comprised within the scope of the present invention. Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims.

All publications and references are herein expressly incorporated by reference in their entirety. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A method of reducing and/or protecting against disorders associated with neuronal cell damage in a perinatal subject in need thereof, comprising:
administering a therapeutically effective amount of neuregulin (NRG) and a pharmaceutically acceptable carrier to the perinatal subject.

2. The method of claim 1, wherein NRG is neuregulin-1 (NRG-1).

3. The method of claim 1, wherein the perinatal subject is a neonate born prior to 32 weeks of gestation.

4. The method of claim 1, where in the step of administering further comprises administering neuregulin to a pregnant subject.

5. The method of claim 1, wherein the perinatal subject is a fetus.

6. The method of claim 1, wherein the method further comprises increasing ErbB-receptor expression.

7. The method of claim 1, wherein the neuregulin is administered in conjunction with an endogenous protector.

8. The method of claim 7, wherein the endogenous protector is selected from the group consisting of glucocorticoids and thyroid hormones.

9. The method of claim 1, wherein the neuregulin is administered in conjunction with an exogenous protector.

10. The method of claim 1 wherein the step of administering the therapeutically effective amount of NRG is at least one of an oral administration, a parenteral administration, an intravenous administration, an intramuscular administration, an intraamniotic administration, a sub-cutaneous administration, a transdermal administration, an intratechal administration, a rectal administration, intravaginal administration, intra peritoneal or amniotic administration, and an intranasal administration.

11. A method of assessing whether a perinatal subject is at risk for developing a neurological disorder associated with neuronal cell damage, the method comprising:
evaluating levels of neuregulin wherein a reduced level or lack of is an indication that the perinatal subject is at risk for developing the disorder.

12. The method of claim 11, wherein the perinatal subject is a neonate born prior to 32 weeks of gestation.

13. The method of claim 11, wherein the perinatal subject is a fetus.

14. The method of claim 11, wherein the step of evaluating comprises measuring expression levels of neuregulin.

15. The method of claim 14, wherein the step of measuring further comprises measuring expression levels of ErbB receptors.

16. The method of claim 14, wherein the step of measuring comprises measuring levels of at least one of a ribonucleic acid, a deoxynucleic acid and a protein.

17. The method of claim 11, wherein evaluating levels further comprises analyzing neuregulin for one or more single nucleotide polymorphisms.

18. The method of claim 17, wherein the one or more polymorphisms comprise SNP8NRG221533.

19. The method of claim 11, wherein the step of evaluating levels comprises measuring activation levels of neuregulin signaling pathways.

20. The method of claim 11, wherein the step of evaluating levels further comprises analyzing at least one ErbB receptor gene for one or more single nucleotide polymorphisms.

21. The method of claim 11, wherein the disorder is at least one of cerebral palsy and mental retardation.

22. A method of evaluating a risk of a NRG-1 deficiency in a perinatal subject, comprising the steps of:
assaying a sample from the perinatal subject for a polymorphism associated with decreased expression of NRG-1.

23. The method of claim 22, wherein the perinatal subject is a fetus.

24. The method of claim 22, wherein the step of assaying the sample further comprises obtaining the sample from a fetus in a pregnant subject.

25. The method of claim 22, wherein the perinatal subject is a neonate.

26. A method of evaluating risk of a neuregulin deficiency in a perinatal subject comprising:
providing a nucleic acid sample from the perinatal subject;
determining a single nucleotide polymorphism (SNP) genotype; and
comparing the SNP genotype with a predetermined SNP genotype, whereby the perinatal subject is predicted to be at risk of a neuregulin deficiency if the SNP genotype comprises at least one of a SNP8NRG221132, a SNP8NRG221533, a SNP8NRG241930, and a SNP8NRG433E1006.

27. A method of diagnosing or predicting risk of a neuregulin deficiency in a perinatal subject comprising:
determining a presence or absence of a neuregulin polymorphism, wherein the polymorphism is at least one of a SNP8NRG221132, a SNP8NRG221533, a SNP8NRG241930, SNP8NRG243177 and a SNP8NRG433E1006.

28. The method of claim 27, wherein determining the presence or absence comprises enzymatic amplification of nucleic acid from the perinatal subject.

29. The method of claim 28, wherein determining the presence or absence of a polymorphism further comprises restriction fragment length polymorphism analysis.

30. The method of claim 28, wherein determining the presence or absence of a polymorphism further comprises sequence analysis.

31. The method of claim 27, wherein the method further comprises determining the presence of a SNP8NRG221533 polymorphism.
